(19) United States
(12) Patent Application Publication
(10) Pub. No.: US 2017/0128615 A1
(43) Pub. Date: May 11, 2017

(54) Transposable Pressure Sensitive Adhesives, Articles, and Related Methods

(71) Applicant: Avery Dennison Corporation, Glendale, CA (US)

(72) Inventors: Eric L. BARTHOLOMEW, Mill Hall, PA (US); Kyle R. HEIMBACH, Millmont, PA (US); Christopher E. KOHLER, S. Williamsport, PA (US); Michael ZAJACZKOWSKI, Bellefonte, PA (US)

(21) Appl. No.: 15/319,255

(22) PCT Filed: Jun. 18, 2015
(86) PCT No.: PCT/US2015/036322

§ 371 Date: Dec. 15, 2016

Related U.S. Application Data
(60) Provisional application No. 62/014,039, filed on Jun. 18, 2014.

(51) Int. Cl. A61L 15/58; C09J 7/02; A61F 13/02; C09J 153/00

(52) U.S. Cl. CPC A61L 15/58; C09J 153/00; C09J 7/0225; C09J 7/0221; A61F 13/0253; A61F 13/0256; C09J 2453/00; C09J 2205/114

(57) ABSTRACT

Transposable adhesives which exhibit a change in adhesive properties upon exposure to particular stimuli or environmental factors are described. The adhesives include one or more olefin block copolymers in combination with tackifiers and process oils. The transposable adhesives may additionally include one or more polyolefin elastomers.

### CROSS-REFERENCE TO RELATED APPLICATION

The present application is a 371 of International Patent Application No. PCT/US2015/036322, which was published in English on Dec. 23, 2015, and claims the benefit of U.S. Provisional Patent Application No. 62/014,039 filed Jun. 18, 2014, both of which are incorporated herein by reference in their entireties.

### FIELD

The present subject matter relates to transposable pressure sensitive adhesives and particularly such adhesives utilizing olefin block copolymers. The adhesives find wide application and particularly as medical adhesives. The present subject matter also relates to adhesive articles using the transposable adhesives, and methods of use of the adhesives and articles.

### BACKGROUND

Pressure sensitive adhesives (PSAs) have been used in many industries that require bonding of two or more materials. PSAs are commercially available in many forms such as for example as hot/warm melts, solvent borne formulations, water based formulations, and syrups. PSAs are used in applications as simple as paper labels to high performance tapes used to bond components in automobiles. Regardless of the application, PSAs typically exhibit a set of viscoelastic properties that provide prolonged adhesion characteristics once applied to a substrate. These adhesion characteristics are relatively constant, other than typical responses from degradation due to external environmental conditions such as temperature and chemical exposure, for example.

With advancements in technology over the past decade, applications now exist that require PSAs to exhibit different adhesion performances at different stages of application. An example is a PSA that exhibits properties suitable for a removable label at a first application stage, but which transposes to a high strength PSA similar to an HVAC tape in a second stage, when activated by an external stimuli. This concept extends beyond mere transposition from one PSA state to another, but also includes transpositions from a PSA state to a structural bond state. Additional applications include the need for transposition from high adhesion to low adhesion, i.e., “debond on demand,” that has been heavily researched and well documented.

Particularly, a need has emerged within the medical industry that requires an adhesive with high adhesive properties that can also be easily removed. For example, people often use a bandage when they accidentally acquire a scrape or cut. Current market demands require the bandage to be waterproof, sweat proof, and be sufficiently flexible to move with a user throughout the period of use without incident while the wound heals. Periodically, the user will change or remove the bandage. However, in order for the bandage to stay in place, the adhesive needs to be relatively aggressive. This is acceptable until the time of removal. While some people may use a bandage infrequently such as once every several months, for others bandage use may be an everyday ritual. For example, those with ostomy or stoma apparatus need to change a rather large bandage on a daily basis. Accordingly, this is an example of a need for a transposable adhesive which selectively exhibits different adhesion characteristics at different times or stages.

### SUMMARY

The difficulties and drawbacks associated with previous approaches are addressed in the present subject matter as follows.

In one aspect, the present subject matter provides a transposable adhesive comprising at least one olefin block copolymer, at least one tackifier, and at least one process oil or extender. Upon exposure to one or more stimuli or environmental factors selected from the group consisting of (i) changes in temperature, (ii) changes in pressure, (iii) exposure to at least one chemical agent, (iv) exposure to light, and (v) combinations of (i)-(iv), at least one adhesive property of the adhesive changes.

In another aspect, the present subject matter provides an adhesive article including a substrate and disposed on the substrate, a transposable adhesive. The adhesive comprises at least one olefin block copolymer, at least one tackifier, and at least one process oil or extender. Upon exposure to one or more stimuli or environmental factors selected from the group consisting of (i) changes in temperature, (ii) changes in pressure, (iii) exposure to at least one chemical agent, (iv) exposure to light, and (v) combinations of (i)-(iv), at least one adhesive property of the adhesive changes.

In yet another aspect, the present subject matter provides a method of improving ease of removal of an adhered article from a surface. The method comprises providing an article to be adhered to a surface. The method also comprises providing a region of a transposable adhesive between the article and the surface. The adhesive includes (i) at least one olefin block copolymer, (ii) at least one tackifier, and (iii) at least one process oil or extender, and upon exposure to one or more stimuli or environmental factors selected from the group consisting of (a) changes in temperature, (b) changes in pressure, (c) exposure to at least one chemical agent, (d) exposure to light, and (e) combinations of (a)-(d), the adhesive strength of the adhesive decreases. The method also comprises adhering the article to the surface. Prior to removal of the article adhered to the surface, the transposable adhesive is exposed to one or more of (a)-(e), thus resulting in a decrease of the adhesive strength of the adhesive and thereby improving ease of removal of the adhered article from the skin.

As will be realized, the subject matter described herein is capable of other and different embodiments and its several details are capable of modifications in various respects, all without departing from the claimed subject matter. Accordingly, the drawings and description are to be regarded as illustrative and not restrictive.

### BRIEF DESCRIPTION OF THE DRAWINGS

FIG. 1 is a graph illustrating an overall viscoelastic window for all pressure sensitive adhesives.

FIG. 2 is a graph illustrating curing of traditional pressure sensitive adhesives and structural adhesives as compared to a representative transposable adhesive.

FIG. 3 is a graph illustrating viscoelastic windows measured for another representative transposable pressure sensitive adhesive and described in Example 3 before and after transposition.

FIG. 4 is a graph illustrating storage modulus as a function of temperature for representative transposable pressure sensitive adhesives described in Examples 2 and 3.

FIG. 5 is a graph illustrating storage modulus and peel as a function of temperature for a transposable pressure sensitive adhesive according to the present subject matter.

### DETAILED DESCRIPTION OF THE EMBODIMENTS

Pressure sensitive adhesives (PSAs) are used in many different applications in which the performance properties of the adhesive are specific and constant as applied. However, recent increasing performance demands require performance windows beyond typical PSA properties after application. Adhesives that can go from one PSA state to another, once applied, provide an opportunity to expand performance in situ. One example is a removable adhesive that can transpose to a permanent adhesive after a triggered reaction. The present subject matter describes a fundamental characterization of PSAs via viscoelastic properties. In addition, routes to acrylic PSA systems that transpose from one set of properties once applied to another set of properties with supporting examples thereof are described. The examples focus on specific applications where high strength adhesives are needed to withstand harsh environments that standard PSAs cannot tolerate.

Specifically, the present subject matter provides pressure sensitive adhesives (PSA) that transpose from low adhesion properties to high adhesion properties via precise thermal- or UV-activated chemistries. The term “transposable adhesive” as used herein refers to an adhesive which changes state from exposure to stimuli and in certain embodiments an adhesive having adhesive properties or characteristics that change upon exposure to one or more stimuli or environmental factors such as (i) changes in temperature and particularly heating, (ii) changes in pressure, (iii) exposure to one or more chemical agents, (iv) exposure to light and particularly UV light, and (v) combinations of (i)-(iv). In many embodiments of the present subject matter, the transposable adhesive exhibits a change in adhesive characteristics upon heating, and particularly an increase in adhesive strength upon heating. And, in particular embodiments, the transposable adhesive exhibits a change in adhesive characteristics upon heating to a temperature of at least about 25° C., and typically at least about 30° C., and particularly at least 32° C. The human body has a typical skin surface temperature of 35° C. Thus, in many embodiments of the present subject matter, the transposable adhesive is configured to exhibit optimum adhesive properties around 35° C., such as for example adhesive strength and retention.

The adhesion properties of a PSA can be manipulated by multiple variables during the synthesis of the polymer. In Table 1 below, several contributing variables, along with their property effects, are presented.

TABLE 1
Synthesis Variables and Their Resulting Property Effects

| Variable | Effect |
|---|---|
| Monomer Choice | Glass Transition, Solubility Parameter |
| Functionality of Monomers | Adhesion Promotion, Crosslinking Sites |
| Molecular Weight | Peel/Shear Balance |
| Branching | Cohesive Strength |

In addition to synthesis variables, particular additives can be introduced to the polymer post-synthesis that enhance certain adhesion properties of the PSA. Representative examples of these additives are shown in Table 2.

TABLE 2
Additives and Their Resulting Property Effects

| Additive | Effect |
|---|---|
| Tackifier | Improve Tack/Peel |
| Crosslinker | Improve Cohesive Strength |
| Fillers | Resistance (i.e., Flame, Chemical, etc.) |
| Antioxidants | Oxidative Degradation |

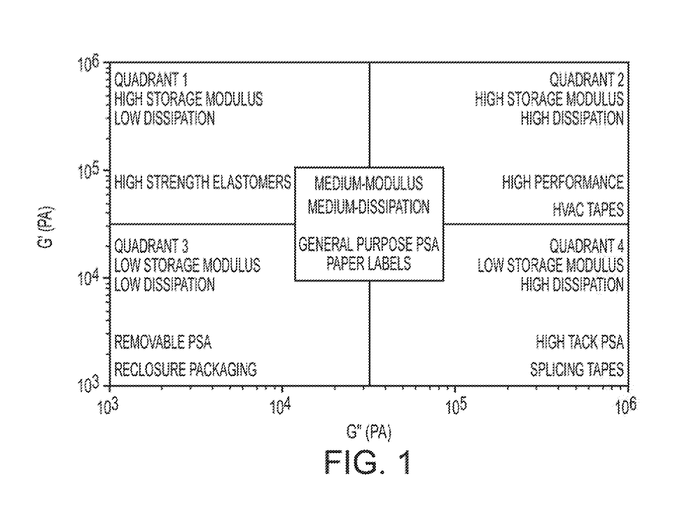

With the combination of the synthesis variables and additives, a broad range of PSA performance can be achieved. Performance can be qualified by the viscoelastic properties of the PSA. Dynamic Mechanical Analysis (DMA) enables the industry to measure the viscoelastic properties of PSAs. In this technique, the storage (G′) and loss (G″) moduli are measured at two frequencies which represent the bonding and debonding states. With the corresponding measurements, the PSA performance window of an adhesive can be predicted based on where the measured moduli fall on the plot depicted in FIG. 1. All PSAs will have a performance window somewhere in FIG. 1.

Using this method of characterization, data can confirm the transposition of PSAs from one set of PSA performance to another, i.e., from one quadrant or region to another. Data can also suggest a PSA transposing from one PSA quadrant to a structural state located well above the Dahlquist criteria (G′=10⁵). The Dahlquist criteria are described in Pocius, A.V., “Adhesion & Adhesives: An Introduction,” Hanser Publications, New York, N.Y., First Edition, (1997); and “Handbook of Pressure Sensitive Adhesive Technology,” Edited by D. Satas, p. 172, (1989).

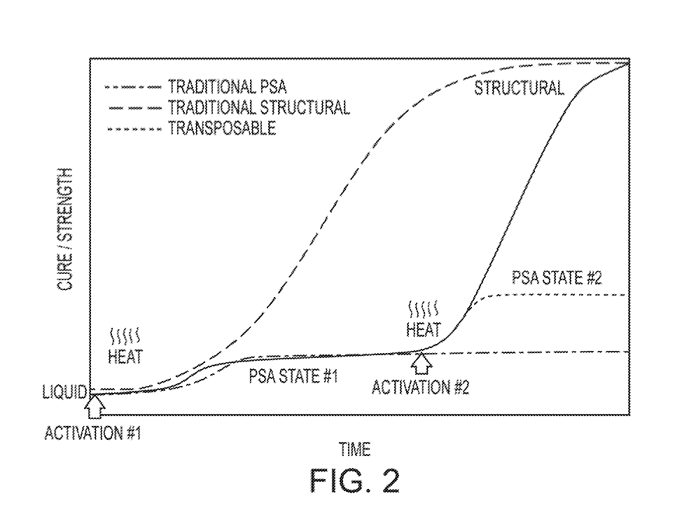

In certain embodiments of the present subject matter, the adhesives are transposable upon exposure to heat or UV radiation. Such adhesives use a thermal or UV activator that triggers a chemical reaction to cause the adhesion properties to change after application. Whether the change in adhesion properties is to another PSA type or to a structural bond state, is dependent on the application. For the examples described herein, the transposition chemistry is epoxy polymerization via the activators. FIG. 2 shows the transposition process as a function of cure/strength vs. time.

Referring to FIG. 2, activation #1 designates a coating process or mixing of 100% solids of a two-part structural adhesive which yields a PSA of state #1 or structural state respectively. At this point, general crosslinking present in the base polymer system is contributing to the PSA performance, but not the epoxy chemistry. Activation #2 occurs sometime after application and designates the triggerable reaction to cause the transposition to PSA state #2 or structural. After activation #1, there is no change in performance of state #1 after application until activation #2. In comparison, traditional PSA and structural adhesives have only one activation (such as for example which occurs at coating/drying PSAs or mixing two-part structural adhesives) and directly reach their end state. Transposable adhesives allow a user to take advantage of PSA state #1 for extended periods of time and activate to state #2 on demand by either thermal or UV exposure.

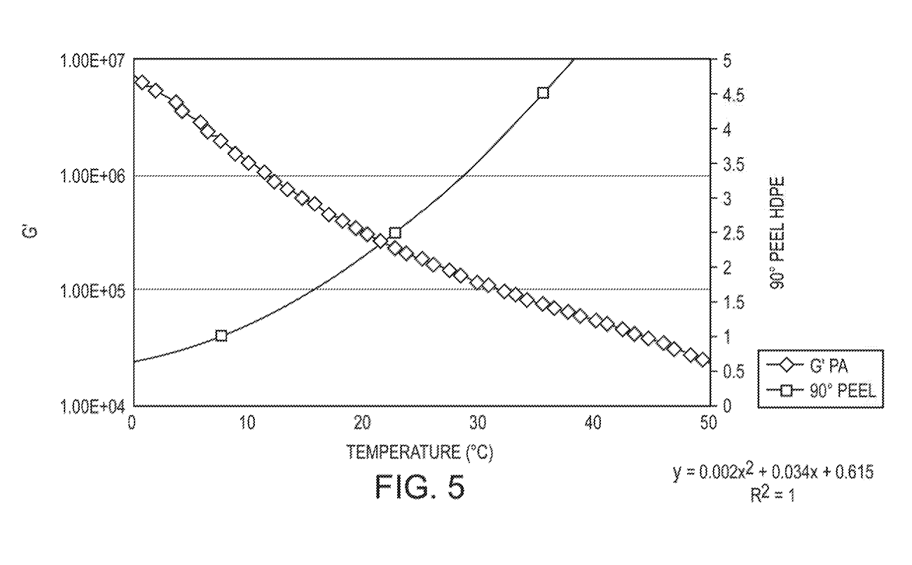

In certain embodiments of the present subject matter, the transposable adhesives are configured to increase in G′ modulus as temperature decreases, and particularly to also exhibit increased peel strengths as temperature increases. These behaviors are shown in FIG. 5.

In particular versions of the present subject matter, transposable adhesives are provided that undergo a change in adhesive characteristics upon exposure to heat. In certain embodiments, the change in adhesive characteristics includes an increase in adhesive strength. And, in certain embodiments, the change in adhesive characteristics occurs upon heating to a temperature of at least about 30° C., and particularly at least 32° C.

The transposable adhesives of the present subject matter comprise (i) one or more olefin block copolymer(s), (ii) one or more tackifiers, and (iii) one or more process oils or extenders. These transposable adhesives may optionally also comprise one or more polyolefin elastomers. It is also contemplated that a variety of optional additives can also be incorporated into the adhesives.

An array of olefin block copolymer(s) can be used in the transposable adhesives of the present subject matter. Generally, the olefin block copolymers are polyolefins with alternating blocks of hard, i.e., relatively rigid, and soft, i.e., highly elastomeric, segments. Typically, the olefin block copolymers used in the noted transposable adhesives exhibit a melt index as measured by ASTM D1238 within a range of from 10 to 20 g/10 min (2.16 kg @ 190° C.), and particularly 15 g/10 min (2.16 kg @ 190° C.). The noted olefin block copolymers have a density within a range of 0.86 to 0.88, and particularly 0.866 to 0.877, and in certain embodiments 0.866 g/cm³. The noted olefin block copolymers exhibit a Shore A hardness as measured by ASTM D2240 within a range of 50 to 75, particularly from 50 to 60, and in certain versions 55. The noted olefin block copolymers exhibit a tensile modulus 100% Secant as measured by ASTM D638 within a range of 150 to 350 psi, particularly from 150 to 200 psi, and in certain versions 189 psi. The noted olefin block copolymers exhibit an ultimate tensile strength as measured by ASTM D638 within a range of from 150 to 400 psi particularly from 150 to 200 psi, and in certain versions 176 psi. The noted olefin block copolymers exhibit an ultimate tensile elongation as measured by ASTM D638 within a range of from 1,000 to 1,800%, particularly from 1,000 to 1,400%, and in certain versions 1,200%. The noted olefin block copolymers exhibit an ultimate tensile strength as measured by ASTM D412 within a range of from 400 to 1,200 psi, particularly from 400 to 450 psi, and in certain versions 435 psi. The noted olefin block copolymers exhibit an ultimate tensile elongation as measured by ASTM D412 within a range of 1,500 to 2,300%, particularly from 2,000 to 2,200%, and in certain versions 2,200%. The noted olefin block copolymers exhibit a tear strength as measured by ASTM D624 within a range of 15 to 35 kN/m, particularly from 15 to 20 kN/m, and in certain versions 17 kN/m. The noted olefin block copolymers exhibit a melting temperature as measured by differential scanning calorimetry (DSC) within a range of 230 to 260° F. (110 to 127° C.), particularly from 240 to 250° F. (116 to 121° C.), and in certain embodiments 244° F. (118° C.).

A particular olefin block copolymer which is commercially available and which can be used in the transposable adhesives of the present subject matter is INFUSE 9807 available from Dow Chemical. Table 3 set forth below lists various properties of the INFUSE 9807 olefin block copolymer.

TABLE 3
Properties of INFUSE 9807 Olefin Block Copolymer

| | Nominal | | Nominal | | |
|---|---|---|---|---|---|
| | Value | | Value | | Test |
| Physical | (English) | | (SI) | | Method |
| Density | 0.866 | g/cm³ | 0.866 | g/cm³ | ASTM D792 |
| Melt Index | 15 | g/10 min | 15 | g/10 min | ASTM D1238 |
| (190° C./2.16 kg) | | | | | |
| Tensile Modulus- | 189 | psi | 1.30 | MPa | ASTM D638 |
| 100% Secant | | | | | |
| (Compression Molded) | | | | | |
| Tensile Strength | 176 | psi | 1.21 | MPa | ASTM D638 |
| (Break, Com- | | | | | |
| pression Molded) | | | | | |
| Tensile Elongation | 1,200% | | 1,200% | | |
| Break, | | | | | |
| Compression Molded | | | | | |
| Tensile Strength | 435 | psi | 3.00 | MPa | ASTM D412 |
| (Break) | | | | | |
| Tensile Elongation | 2,200% | | 2,200% | | ASTM D412 |
| (Break) | | | | | |
| Tear Strength | 97.1 | lbf/in | 17.0 | kN/m | ASTM D624 |
| Compression Set | | | | | |
| 70° F. (21° C.) | 16% | | 16% | | ASTM D395 |
| 158° F. (70° C.) | 76% | | 76% | | |
| Durometer Hardness | | | | | |
| Shore A, | 55 | | 55 | | ASTM D2240 |
| Compression Molded | | | | | |
| Melting | 244° | F. | 118° | C. | Dow Method |
| Temperature (DSC) | | | | | |

A variety of tackifiers can be used in the adhesives of the present subject matter. Nonlimiting examples of such tackifiers include terpene resins, low molecular weight hydrogenated hydrocarbons, and combinations thereof. An example of a suitable terpene resin is SYLVARES TR M1115 available from Arizona Chemical. An example of a suitable hydrogenated hydrocarbon is H-100L available from Eastman Chemical.

Various oils or extending agents may also be present in the adhesive compositions. The above broadly includes not only the usual plasticizing oils but also contemplates the use of olefin oligomers and low molecular weight polymers as well as vegetable and animal oil and their derivatives. Petroleum derived oils may be employed, and are typically relatively high boiling materials containing only a minor proportion of aromatic hydrocarbons (typically less than 30% and, more particularly, less than 15% by weight of the oil). Alternatively, the oil may be totally non-aromatic. The oligomers may be polypropylenes, polybutenes, hydrogenaged polyisoprene, hydrogenated polybutadiene, or the like, having average molecular weights between about 350 and about 10,000. Vegetable and animal oils include glyceryl esters of the usual fatty acids and polymerization products thereof. Nonlimiting examples of suitable oils include BVA 100 process oil from BVA Inc., and AD500 process oil available from Calumet Specialty Products. Combinations of any of these may be used.

As previously noted, in certain embodiments the transposable adhesives also comprise one or more polyolefin elastomers. Typically, in many embodiments the polyolefin elastomers are copolymers of ethylene and another alpha-olefin such as for example butane or octane. The polyolefin elastomers typically have a density within a range of from 0.865 to 0.880, particularly from 0.865 to 0.875, and in certain versions 0.870 g/cm³. The polyolefin elastomers typically have a Brookfield viscosity at 350° F. (177° C.) measured by ASTM D1084, within a range of from 6,500 to 18,000 cps, more particularly from 8,000 to 8,500 cps, and in certain versions 8,200 cps. The polyolefin elastomers typically have a melt index within a range of 400 to 1,500, particularly 800 to 1,200, and in certain versions 1,000 g/10 min (190° C., 2.16 kg). The polyolefin elastomers typically have a DSC melting point within a range of 150 to 160° F., particularly from 152 to 156° F., and in certain versions 154° F. The polyolefin elastomers typically have a crystallinity within a range of from 14 to 24%, particularly from 14 to 18%, and in certain versions 16%. The polyolefin elastomers typically exhibit a tensile strength as measured by ASTM D638 of from 200 to 250 psi, particularly from 210 to 240 psi, and in certain versions 225 psi. The polyolefin elastomers typically exhibit a tensile elongation (break) as measured by ASTM D638 within a range of from 80% to 150%, particularly from 100% to 120%, and in certain embodiments 110%. The polyolefin elastomers typically have a glass transition temperature within a range of from −75 to −65° F. (−60 to −54° C.), particularly from −74 to −70° F. (−59 to −57° C.), and in certain versions −72° F. (−58° C.).

An example of a suitable polyolefin elastomer is AFFINITY GA 1900 which is commercially available from Dow Chemical. Table 4 set forth below lists various properties of AFFINITY GA 1900.

TABLE 4
Properties of AFFINITY GA 1900 Polyolefin Plastomer

| | Nominal Value | | | | |
|---|---|---|---|---|---|
| Physical | (English) | | Nominal Value (SI) | | Test Method |
| Density | 0.870 | g/cm³ | 0.870 | g/cm³ | ASTM D792 |
| Gardner Color | <2.00 | | <2.00 | | ASTM D6290 |
| Volatile Matter | <0.15% | | <0.15% | | ASTM D3030 |
| Tensile Strength | 225 | psi | 1.55 | MPa | ASTM D638 |
| Tensile Elongation (Break) | 110% | | 110% | | ASTM D638 |
| Glass Transition Temperature | −72.0° | F. | −57.8° | C. | |
| Melting Temperature (DSC) | 154° | F. | 67.8° | C. | |
| Brookfield Viscosity | 8.20 | Pa • s | 8.20 | Pa • s | ASTM D1084 |
| (350° F. (177° C.)) | | | | | |

The adhesives can optionally comprise one or more additives such as oils, antioxidants or stabilizers, antimicrobial agents, pigments, fibers, solvents, and combinations thereof.

Table 5 set forth below lists typical and particular proportions by weight of each of the noted components of the transposable adhesives of the present subject matter.

TABLE 5
Proportions of Components in Transposable Adhesives

| | Weight Proportion | |
|---|---|---|
| Component | Typical | Particular |
| Olefin Block Copolymer(s) | 5-25% | 10-20% |
| Polyolefin Elastomer(s) (Optional) | 10-30% | 20-25% |
| Tackifier(s) | 40-65% | 45-60% |
| Process Oil(s) | 15-35% | 18-30% |

The adhesive compositions are prepared by blending the components in a melt at a temperature of about 130° to 200° C. (about 266° to 392° F.) until a homogeneous blend is obtained, which typically occurs at approximately two hours. Various methods of blending are known to the art and any method that produces a homogeneous blend is satisfactory.

The present subject matter also provides adhesive articles using the transposable adhesives described herein. The adhesive articles include a continuous or discontinuous adhesive layer, typically a transposable pressure sensitive adhesive layer, disposed on a substrate or backing of the article. The adhesive layer typically has a thickness from about 10 to about 125, or from about 25 to about 75, or from about 10 to about 50 microns. In one embodiment, the coat weight of the pressure sensitive adhesive is in the range of about 10 to about 50 grams per square meter (gsm), and in one embodiment about 20 to about 35 gsm. One or more release liners can be used to cover the otherwise exposed regions or faces of the adhesive on the article.

The pressure sensitive adhesive and particularly the transposable pressure sensitive adhesive, can be applied using standard coating techniques, such as curtain coating, gravure coating, reverse gravure coating, offset gravure coating, roller coating, brushing, knife-over roll coating, air knife coating metering rod coating, reverse roll coating, doctor knife coating, dipping, die coating, spraying, and the like. The application of these coating techniques is well known in the industry and can effectively be implemented by one skilled in the art. The knowledge and expertise of the manufacturing facility applying the coating determine the preferred method. Further information on coating methods can be found in “Modern Coating and Drying Technology”, by Edward Cohen and Edgar Gutoff, VCH Publishers, Inc., 1992.

Release liners for use in the present subject matter may be those known in the art. In general, useful release liners include polyethylene coated papers with a commercial silicone release coating, polyethylene coated polyethylene terephthalate films with a commercial silicone release coating, or cast polypropylene films that can be embossed with a pattern or patterns while making such films, and thereafter coated with a commercial silicone release coating. In certain embodiments, a release liner is kraft paper which has a coating of low density polyethylene on the front side with a silicone release coating and a coating of high density polyethylene on the back side. Other release liners known in the art are also suitable as long as they are selected for their release characteristics relative to the pressure sensitive adhesive chosen for use in the present subject matter.

Nonlimiting examples of adhesive articles using the transposable adhesives include, but are not limited to tapes and particularly medical and surgical tapes which can be single or dual sided, bandages, dressings, wound coverings, ostomy components including ostomy appliances and stoma components, devices and sensors that are adhered or otherwise contacted with skin such as biosensors.

The pressure sensitive adhesive article of the present subject matter may be used in a wide variety of applications such as adhesive articles for medical use including bandages, surgical drapes, intravenous dressings, wound dressings, and self adhesive wound rolls. Additional applications include industrial, automotive, aerospace, military or consumer use such as floor covering adhesives, shock absorbent adhesive mounts, double sided adhesive articles, self adherent labels, self sealing envelopes, resealable bags, envelopes and containers, single and double faced adhesive tape, weather-stripping, thermal insulation, and sound insulation.

As described herein, in many embodiments, the transposable adhesives significantly increase in adhesive strength and/or retention upon heating to a temperature associated with biological skin. And conversely, upon a reduction in temperature, the transposable adhesives undergo a decrease in adhesive strength and/or retention. Thus, for adhesive articles using such transposable adhesives and which are adhered to biological skin, removal of the article, i.e., debonding of the adhesive from the skin, can be facilitated by reducing the temperature of the adhesive article. This can conveniently be performed by applying a cold compress or ice pack for example to the adhesive or adhesive article. After the temperature has been reduced, the article can be easily removed with little or no discomfort.

Thus, the present subject matter also provides various methods and techniques in which the removal of adhesive articles from a surface, for example biological skin, can be facilitated by use of the transposable adhesives described herein. For example, in one embodiment, a method for improving ease of removal of an adhered article is provided in which the adhesive used to adhere the article to the surface of interest is a transposable adhesive as described herein. If the transposable adhesive is selected or otherwise configured to exhibit a reduction in adhesive bonding upon temperature reduction, the removal method simply involves reducing the temperature of the adhesive. Such temperature reductions can be readily performed by contacting the adhesive article with a cold pack or other cooling component.

### EXAMPLES

Examples and data are described which demonstrate the transposition of adhesion properties of a base acrylic polymer using various activating chemistries. The examples are evaluated in tape form and are designed for tape applications. For structural adhesive applications, improved tape stability enables the adhesive to be supplied in tape form for easy and efficient application through controlled bond lines, which is an advantage over a less favorable approach of using applicator guns.

The same base solution polymer was used for all the examples as noted in which the examples only differ by the amount of epoxy diluent added. Base acrylic esters such as 2-ethylhexyl acrylate (EHA), butyl acrylate (BA), and acrylic acid (AA) were obtained from various commercial suppliers and used as received to polymerize the base polymer. The acrylic polymerization was initiated with azobis(isobutyronitrile) (AIBN) and made in organic solvents. The base polymer was formulated with aluminum acetoacetonate (AAA) at various levels by weight based on polymer solids. Epoxy S-21 was obtained from Synasia and used as delivered. The thermal super acid generator was obtained from King Industries and used as received. All samples were coated at approximately 2.0 mil adhesive thicknesses onto 100% solids platinum-cured silicone paper liner. The coatings were all air dried for 10 minutes and placed in a forced air oven in 10 minutes at 80° C. and closed with 100% solids platinum cured silicone paper liner. Adhesion testing was done on transfer coatings to 2.0 mil aluminum foil. The laminates were all aged in a controlled climate room (70° F. at 50% humidity) for 24 hours prior to testing. The transposition was done via thermal activation of epoxy polymerization at 140° C. for 15 minutes after application by the designated dwell time. The UV example was transposed via an Uvitron Intella-ray unit fitted with 600 watt/in UV-B bulb. The adhesive was exposed to UV light and then immediately used to construct an aluminum lap shear such that the transposition chemistry was initiated but completely after the lap shear construction was made.

Dynamic Mechanical Analysis (DMA) was performed on a TA Instrument AR-2000 rheometer using parallel plate clamps. 1.0 mm thick samples were placed in the clamp and equilibrated to 30° C. Frequency sweeps were conducted from 0.01 rad/s to 100 rad/s at 30° C. to construct the viscoelastic windows. Temperature sweeps were conducted from −80° C. to 180° C. to measure storage modulus at 10 rad/s.

### Results and Discussion

The first example, i.e., Example 1, is shown in Table 6, where the adhesive system transposes from a low strength adhesive to a structural end state via UV activation. The tensile strength is reported for a 1 inch by 1 inch overlapped aluminum lap shear (ASTM D1002) for the base PSA, a high performance PSA modified with reactive silane oligomer to form a high strength interpenetrating network (IPN)9, and the base PSA transposed to structural.

TABLE 6
Tensile Strength of Transposed Adhesive vs. the Base
Adhesive and a High Strength IPN PSA

| | Base PSA | IPN PSA | Transposed PSA |
|---|---|---|---|
| 1″ × 1″ Aluminum Foil | 45 | 290 | 400+ |
| Lap Shear-Tensile (psi) | | | |

The lap shear data shows that the transposable adhesive system improves the tensile strength of the base adhesive by a factor of 10 times and exceeds the tensile strength of a high performance IPN used in industrial tapes (Note: 400 psi was the max for the load cell used in the study). In comparison, high strength structural adhesives have a tensile strength of about approximately 1000 psi (7 MPa). However, some applications only require up to approximately 300 psi (2 MPa) to create a structural bond. The next example used the same base polymer and is transposed to a high-performance type PSA. The 180° peel strength (ASTM D3330) off stainless steel after a 15 minute and 24 hour dwell is reported along with room temperature (ASTM D3654) and 65° C. static shear in Table 7.

TABLE 7
Adhesion Data for the Unreacted Transposable Adhesive vs. the Transposed Adhesive

| | 180° Peel on Stain- | | 8.8 lb/in² Static | 5 lb/in² 65° C. Static Shear on | |
|---|---|---|---|---|---|
| | less Steel (pli) | | Shear on Stainless | Stainless Steel (Min) | |
| Dwell Time | 15 min | 24 hour | Steel (Min) | Min | Slip (mm) |
| Unreacted | 1.97 | 2.38 | 8.55 Adhesive | 78.5 Adhesive | Failure |
| Transposable | | | | | |
| Transposed | 4.34 Stain | 4.47 Stain | 10,000+ | 5,300+ | 0 |
| (Immediate) | | | | | |
| Transposed | 3.80 Stain | 3.91 Stain | 10,000+ | n/a | n/a |
| (24 hr open time) | | | | | |

As reported, the peel adhesion more than doubles after the thermal activation of the epoxy polymerization. Not only does the peel adhesion double, but also the shear dramatically increases. This is unique because typical PSAs have a balancing effect with regards to peel and shear. This effect shows that, as there is an increase in either peel or shear, typically the other property decreases. The enhancements of adhesion and cohesion properties that the transposable adhesive exhibits are also present when the open time (time after application but before epoxy activation) is changed from immediate to 24 hours. Similar adhesion data between these preactivated dwell times suggests this transposable tape system has long/stable open time. This property allows users to apply, remove, and reapply the tape before activation, which makes a permanent bond at the user's will. In addition to increased adhesion, temperature resistance increases with transposition and is supported by the 65° C. shear data included in Table 7. Resistance to temperature makes these tapes attractive for automobile applications under the hood or any application where the adhesive has to withstand high temperatures without degrading.

For Example 2, the transposition was thermally activated at 140° C. for 15 minutes. These conditions may not be acceptable for some applications. To identify the limits of the thermal activation, an investigation was conducted using hot roll lamination on 8 mil aluminum lap shear samples. The rate of the hot roll and the pressure were held constant at 12 in/min (0.30 m/min) and 110 psi respectively, and the temperature of roll was changed. After the samples were transposed via the hot roll lamination step, lap shear peak load was measured to quantify the level transposition. This lap shear data is presented in Table 8 below.

TABLE 8
Tensile Strength vs. Hot Roll Temperature

| Temperature (° F.) | Control (Room Temp.) | 300° F. | 350° F. | 400° F. |
|---|---|---|---|---|
| Peak Load (lbf) | 52.80 | 99.00 | 252.80 | 252.85 |

The data reported is just one example of conditions in which the transposable adhesive was successfully transposed. The level of cure of the activatable epoxy polymerization will be dependent on the substrate and its thickness, and the pressure, speed, and temperature of the hot roll lamination steps. The exact limits of the cure would have to be experimentally found for any given application. For this given construction and hot roll laminator settings, a temperature of 350° F. was sufficient heat to fully transpose the adhesive. The fact that 400° F. yielded the same tensile strength as 350° F. suggests that the max tensile strength (100% cure of epoxy) is roughly 252 lbf.

Another example, i.e., Example 3, is an adhesive that transposes from a removable adhesive to a general purpose PSA using the same base polymer as the previous two examples, and differs only by the amount of epoxy added to the system. The adhesion data is reported in Table 9 below.

TABLE 9
Adhesion Data for the Unreacted Transposable Adhesive vs. the Transposed Adhesive

| | 180° Peel on Stain- | | 8.8 lb/in² Static | 5 lb/in² 65° C. Static Shear on | |
|---|---|---|---|---|---|
| | less Steel (pli) | | Shear on Stainless | Stainless Steel (Min) | |
| Dwell Time | 15 min | 24 hour | Steel (Min) | Min | Slip (mm) |
| Unreacted | 1.04 | 1.31 | 3.05 Adhesive | 2,655 Adhesive | Failure |
| Transposable | | | | | |
| Transposed | 2.16 Stain | 2.30 Stain | 10,000+ | 5,300+ | 0 |
| (Immediate) | | | | | |
| Transposed | 2.29 Stain | 2.51 Stain | 10,000+ | n/a | n/a |
| (24 hr open time) | | | | | |

Comparable to Example 2, the peel adhesion doubles and the cohesion strength dramatically increased, supported by the room temperature and 65° C. shear. Also observed is the enhanced performance independent of the open time as expected from the results of the previous example.

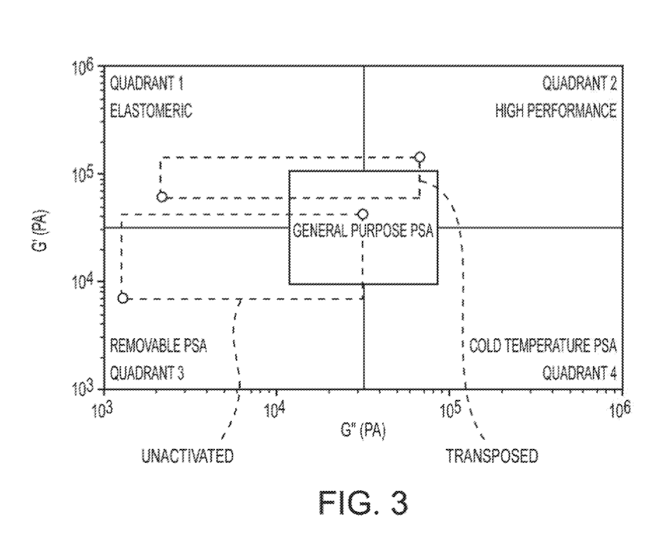

Revisiting the viscoelastic windows mentioned as the qualification of the types of adhesives before and after transposition, Example 3 was examined by DMA. A frequency sweep was conducted in which the performance window was constructed for the unreacted PSA and the transposed PSA. The resulting viscoelastic windows were identified and are displayed in FIG. 3.

As seen in FIG. 3, the performance window was shifted from the removable quadrant to the general purpose region of the overall viscoelastic window plot. Thus, the transposition from one PSA state to another was evident by adhesion performance and analytical techniques.

Higher temperature resistance was included in the adhesion data, but chemical resistance is another property that can also be improved by transposition. To expand on this concept, resistance to SKYDROL was evaluated. SKYDROL is available from Eastman Chemical and is an aviation hydraulic fluid consisting of phosphate esters that is known to degrade polyacrylate elastomers and PSAs. The need for a PSA which resists degradation by this fluid has been an area of focus for the tapes/adhesive industry. The 8 mil aluminum 1 inch by 1 inch lap shears were again used to qualify the transposable adhesive from Examples 2 and 3 before and after being soaked in SKYDROL at 65° C. for 16 hours and are shown in Table 10.

TABLE 10
SKYDROL Resistance Lap Shear Data

| | Control | | Example 2 | | | Example 3 | | |
|---|---|---|---|---|---|---|---|---|
| Transposed | No | No | No | Yes | Yes | No | Yes | Yes |
| Soak | No | Yes | No | No | Yes | No | No | Yes |

Tensile

| | | | | | | | | |
|---|---|---|---|---|---|---|---|---|
| Peak load (lbf) | 69 | 40 | 29 | 373 | 357 | 36 | 350 | 377 |
| Modulus (psi) | 4.02e | 2.17e | 2.25e | 1.65e | 1.68e | 3.36e | 1.57e | 1.54e |

The tensile data reported in Table 10 provides two significant items of information. One, the percentage increase in peak load and modulus for the two examples when being transposed are provided. And two, the percentage decrease in peak load and modulus for the adhesives after the SKYDROL soak are provided. These numbers are shown below in Table 11.

TABLE 11
Percent Increase in Peak Load and Modulus
after Transposition, in Addition to % Decrease
in Peak Load and Modulus after SKYDROL Soak

| | | | Example | Example |
|---|---|---|---|---|
| Process | Measurement | Control | 2 | 3 |
| Trans- | % Increase in Peak Load | — | 439% | 310% |
| position | % Increase in Modulus | — | 405% | 292% |
| After | % Decrease in Peak Load | 43% | 4% | −2% |
| Soak | % Decrease in Modulus | 46% | −8% | 2% |

The degradation of the transposed adhesives was significantly better than the base adhesive, which nearly degraded to 50% of its original strength. In comparison, the transposed adhesives degraded less than 5% if they did not increase in strength. Regarding the values reported in Table 11, negative number for the decrease in peak load or modulus designates that the values increased. For the cases in which there was an increase in value, it was concluded that the percent increase is within the error of the test.

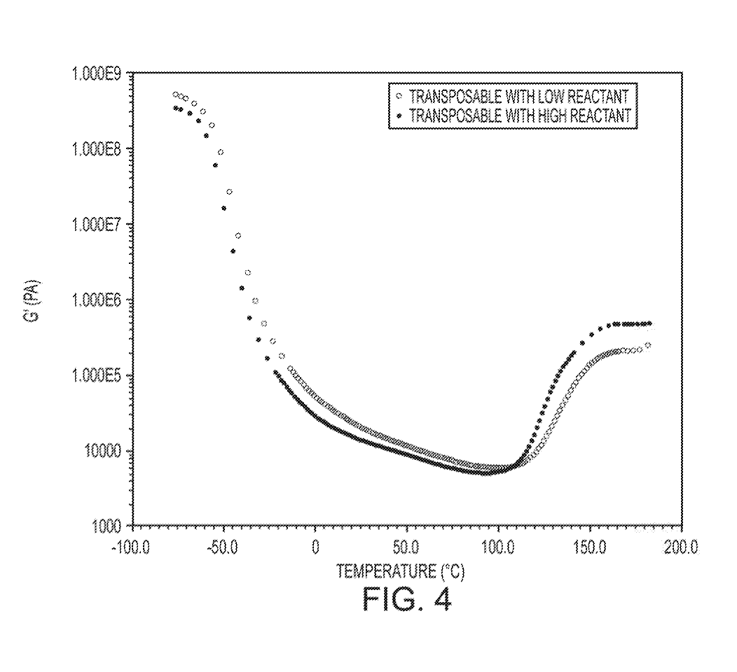

It was observed that the adhesive of Example 3, which had higher epoxy loading, had a lower increase in strength, which is opposite to expectations. To confirm this response to epoxy loading, the storage modulus was measured by a DMA temperature sweep of both examples before transposition. It was expected that higher epoxy loading should result in a lower storage modulus before transposition and a higher storage modulus after transposition. FIG. 4 shows the resulting storage modulus curves for the temperature sweeps and verifies this prediction.

The curve of filled in data points represented the example with higher loading of epoxy. As expected, the storage modulus is lower compared to its lower epoxy loaded example before transposition and higher strength after transposition. This data confirms what was expected, but contradicts the lap shear data in Table 10. The contradiction in the lap shear data was attributed to errors in the lap shear test. Potential causes are lack of adhesion over the entire 1 inch by 1 inch surface due to flexibility of the 8 mil aluminum and possible volume contraction during transposition, leading to lack of adhesion over the entire test area resulting from the lack of flexibility of the aluminum.

Transposable adhesives were characterized physically and analytically by proving a shift of a base adhesive's performance window as a function of storage and loss moduli. The benefit of the shift in performance window was found to be 2 to 3 times increase in peel strength in addition to dramatic increases in cohesive strength after application. The resulting transposed adhesive exhibited enhanced temperature and chemical resistance versus the non-transposed counterpart. It was demonstrated that the transposition process is activated by precise temperatures or UV exposure to yield a tape construction that can be manufactured and applied with long open times before being transposed to high strength or structural adhesive. In tape applications, this is advantageous over traditional two-part structural adhesives due to the lack of applicator guns and controlled bond lines.

In yet another investigation, adhesive samples were prepared as set forth in Table 12. These transposable adhesives are in accordance with the present subject matter. Samples A and B were free of polyolefin elastomer (POE). Samples C and D included polyolefin elastomer. The Samples C and D exhibited easier removability as compared to Samples A and B.

TABLE 12
Embodiments of Transposable Adhesives in Accordance
with Present Subject Matter

| | | | Transposable Adhesives | | | |
|---|---|---|---|---|---|---|
| | | | Without POE | | With POE | |
| | | | Sample A | Sample B | Sample C | Sample D |
| Material | Use | Supplier | % | % | % | % |
| Infuse 9807 (OBC) | Base Polymer PE | Dow | 10 | 20 | 14 | 14 |
| Affinity GA 1900 | Polyolefin | Dow | 0 | 0 | 21 | 21 |
| | Elastomer | | | | | |
| Sylvares TR M1115 | Tackifier | Arizona | 0 | 0 | 46 | 0 |
| H-100L | Tackifier | Eastmen | 60 | 60 | 0 | 46 |
| BVA100 | Process oil | BVA | 0 | 0 | 19 | 0 |
| AD500 | Process oil | Calumet | 30 | 20 | 0 | 19 |

Many other benefits will no doubt become apparent from future application and development of this technology.

All patents, applications, standards, references, and articles noted herein are hereby incorporated by reference in their entirety.

The present subject matter includes all operable combinations of features and aspects described herein. Thus, for example if one feature is described in association with an embodiment and another feature is described in association with another embodiment, it will be understood that the present subject matter includes embodiments having a combination of these features.

As described hereinabove, the present subject matter solves many problems associated with previous strategies, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims.

What is claimed is:

1. A transposable adhesive comprising:
at least one olefin block copolymer;
at least one tackifier; and
at least one process oil or extender;
wherein upon exposure to one or more stimuli or environmental factors selected from the group consisting of (i) changes in temperature, (ii) changes in pressure, (iii) exposure to at least one chemical agent, (iv) exposure to light, and (v) combinations of (i)-(iv), at least one adhesive property of the adhesive changes.

2. The transposable adhesive of claim 1 wherein the one or more stimuli or environmental factors is an increase in temperature.

3. The transposable adhesive of claim 2 wherein the increase in temperature includes heating to a temperature of at least 30° C.

4. The transposable adhesive of claim 1 wherein the at least one adhesive property of the adhesive that changes includes an increase in adhesive strength.

5. The transposable adhesive of claim 1 wherein the olefin block copolymer exhibits a melt index within a range of from 10 to 20 g/10 minutes.

6. The transposable adhesive of claim 1 wherein the olefin block copolymer has a density within a range of 0.86 to 0.88 g/cm³.

7. The transposable adhesive of claim 1 wherein the olefin block copolymer exhibits a melting temperature within a range of 230 to 260° F.

8. The transposable adhesive of claim 1 wherein the olefin block copolymer is present in the adhesive at a weight proportion of 5 to 25%.

9. The transposable adhesive of claim 1 wherein the tackifier is selected from the group consisting of (i) terpene resins, (ii) low molecular weight hydrogenated hydrocarbons, and (iii) combinations of (i)-(ii).

10. The transposable adhesive of claim 1 wherein the tackifier is present in the adhesive at a weight proportion of 40 to 65%.

11. The transposable adhesive of claim 1 wherein the process oil or extender is selected from the group consisting of (i) olefin oligomers, (ii) low molecular weight polymers, (iii) vegetable oils, (iv) animal oils, (v) petroleum oils, and (vi) combinations of (i)-(v).

12. The transposable adhesive of claim 1 wherein the process oil or extender is present in the adhesive at a weight proportion of 15 to 35%.

13. The transposable adhesive of claim 1 further comprising:
at least one polyolefin elastomer.

14. The transposable adhesive of claim 13 wherein the polyolefin elastomer has a density within a range of from 0.865 to 0.880 g/cm³.

15. The transposable adhesive of claim 13 wherein the polyolefin elastomer has a melt index within a range of 400 to 1,500 g/10 minutes.

16. The transposable adhesive of claim 13 wherein the polyolefin elastomer has a melting point within a range of from 150 to 160° F.

17. The transposable adhesive of claim 13 wherein the polyolefin elastomer exhibits a crystallinity within a range of 14% to 24%.

18. The transposable adhesive of claim 13 wherein the polyolefin elastomer exhibits a glass transition temperature within a range of from −75° F. to −65° F.

19. The transposable adhesive of claim 13 wherein the polyolefin elastomer is present in the adhesive formulation at a weight proportion of 10 to 30%.

20. The transposable adhesive of claim 1 further comprising at least one additive selected from the group consisting of (i) oils, (ii) antioxidants or stabilizers, (iii) antimicrobial agents, (iv) pigments, (v) fibers, (vi) solvents, and (vii) combinations of (i)-(vi).

21. An adhesive article including a substrate and disposed on the substrate, a transposable adhesive comprising:
at least one olefin block copolymer;
at least one tackifier; and
at least one process oil or extender;
wherein upon exposure to one or more stimuli or environmental factors selected from the group consisting of (i) changes in temperature, (ii) changes in pressure, (iii) exposure to at least one chemical agent, (iv) exposure to light, and (v) combinations of (i)-(iv), at least one adhesive property of the adhesive changes.

22. The adhesive article of claim 21 wherein the one or more stimuli or environmental factors is an increase in temperature.

23. The adhesive article of claim 22 wherein the increase in temperature includes heating to a temperature of at least 30° C.

24. The adhesive article of claim 21 wherein the at least one adhesive property of the adhesive that changes includes an increase in adhesive strength.

25. The adhesive article of claim 21 wherein the olefin block copolymer exhibits a melt index within a range of from 10 to 20 g/10 minutes.

26. The adhesive article of claim 21 wherein the olefin block copolymer has a density within a range of 0.86 to 0.88 g/cm³.

27. The adhesive article of claim 21 wherein the olefin block copolymer exhibits a melting temperature within a range of 230 to 260° F.

28. The adhesive article of claim 21 wherein the olefin block copolymer is present in the adhesive at a weight proportion of 5 to 25%.

29. The adhesive article of claim 21 wherein the tackifier is selected from the group consisting of (i) terpene resins, (ii) low molecular weight hydrogenated hydrocarbons, and (iii) combinations of (i)-(ii).

30. The adhesive article of claim 21 wherein the tackifier is present in the adhesive at a weight proportion of 40 to 65%.

31. The adhesive article of claim 21 wherein the process oil or extender is selected from the group consisting of (i) olefin oligomers, (ii) low molecular weight polymers, (iii) vegetable oils, (iv) animal oils, (v) petroleum oils, and (vi) combinations of (i)-(v).

32. The adhesive article of claim 21 wherein the process oil or extender is present in the adhesive at a weight proportion of 15 to 35%.

33. The adhesive article of claim 21 wherein the adhesive further comprises:
at least one polyolefin elastomer.

34. The adhesive article of claim 33 wherein the polyolefin elastomer has a density within a range of from 0.865 to 0.880 g/cm³.

35. The adhesive article of claim 33 wherein the polyolefin elastomer has a melt index within a range of 400 to 1,500 g/10 minutes.

36. The adhesive article of claim 33 wherein the polyolefin elastomer has a melting point within a range of from 150 to 160° F.

37. The adhesive article of claim 33 wherein the polyolefin elastomer exhibits a crystallinity within a range of 14% to 24%.

38. The adhesive article of claim 33 wherein the polyolefin elastomer exhibits a glass transition temperature within a range of from −75° F. to −65° F.

39. The adhesive article of claim 33 wherein the polyolefin elastomer is present in the adhesive formulation at a weight proportion of 10 to 30%.

40. The adhesive article of claim 21 wherein the adhesive further comprises at least one additive selected from the group consisting of (i) oils, (ii) antioxidants or stabilizers, (iii) antimicrobial agents, (iv) pigments, (v) fibers, (vi) solvents, and (vii) combinations of (i)-(vi).

41. The adhesive article of claim 21 wherein the article is selected from the group consisting of tapes, bandages, dressings, wound coverings, ostomy components, stoma components, medical devices, sensors, and combinations thereof.

42. A method of improving ease of removal of an adhered article from a surface, the method comprising:
providing an article to be adhered to a surface;
providing a region of a transposable adhesive between the article and the surface, wherein the adhesive includes (i) at least one olefin block copolymer, (ii) at least one tackifier, and (iii) at least one process oil or extender, and upon exposure to one or more stimuli or environmental factors selected from the group consisting of (a) changes in temperature, (b) changes in pressure, (c) exposure to at least one chemical agent, (d) exposure to light, and (e) combinations of (a)-(d), the adhesive strength of the adhesive decreases;
adhering the article to the surface;
whereby prior to removal of the article adhered to the surface, the transposable adhesive is exposed to one or more of (a)-(e), thus resulting in a decrease of the adhesive strength of the adhesive and thereby improving ease of removal of the adhered article from the skin.

43. The method of claim 42 wherein the transposable adhesive undergoes a decrease in adhesive strength upon a reduction in temperature.

44. The method of claim 42 wherein the region of the transposable adhesive is in the form of a layer on the article.

45. The method of claim 42 wherein the surface is biological skin.

46. The method of claim 42 wherein the transposable adhesive also includes at least one polyolefin elastomer.

47. The method of claim 42 wherein the article is selected from the group consisting of tapes, bandages, dressings, wound coverings, ostomy components, stoma components, medical devices, sensors, and combinations thereof.
